(19) United States
(12) Patent Application Publication
(10) Pub. No.: US 2011/0171705 A1
(43) Pub. Date: Jul. 14, 2011

(54) PROCESS FOR THE PRODUCTION OF ALCOHOL

(75) Inventors: Hans Kristian Kotlar, Stavanger (NO); Per Nygård, Stavanger (NO); Mimmi Throne-Holst, Trondheim (NO)

(73) Assignee: STATOIL ASA, Stavanger (NO)

(21) Appl. No.: 13/058,800

(22) PCT Filed: Aug. 14, 2009
(86) PCT No.: PCT/GB2009/002000

§ 371 Date: Mar. 22, 2011

(30) Foreign Application Priority Data
Aug. 14, 2008 (GB) 0814912.2

(51) Int. Cl. C12P 7/04; C12P 7/16; C12N 1/00; C12M 1/00

(52) U.S. Cl. 435/157; 435/160; 435/243; 435/303.1

(57) ABSTRACT

The invention provides a process for the production of an alcohol which comprises cleaving polysaccharides in a cellulosic material in an aqueous medium at a temperature of at least 45° C. using a thermophilic microorganism to yield fermentable sugars, fermenting an aqueous solution of said sugars at a temperature of at least 45° C. with a thermophilic microorganism to yield an alcohol or an alkanoate, if necessary reducing said alkanoate to yield an alcohol, and removing said alcohol from said aqueous solution.

This invention relates to improvements in and relating to a process for the production of alcohol from a cellulosic material which may be run at elevated temperature.

Alcohols, or more precisely C₁₋₆ monohydric alkanols, especially ethanol and butanol, are of increasing importance as fuels, either as such or as additives to conventional hydrocarbon fuels such as gasoline. Alcohols may be produced by fermentation of sugars, such as pentoses and hexoses, derived from plant material. While currently much emphasis has been on the use of plant seeds, e.g. maize, as the raw plant material, this is relatively undesirable as such seed material may alternatively serve as food for human or animal consumption. There is thus a desire to use instead cellulosic material which is unsuitable for human consumption, e.g. wood pulp, forest debris, paper, grass, straw, corn husks, etc. For such raw materials, the cellulose and hemicellulose polysaccharides (hereinafter jointly referred to as cellulose for convenience) must first be broken down to fermentable sugars, generally hexoses and pentoses, whereafter those sugars may be fermented (metabolized) by microorganisms to yield alcohols. Yeasts, e.g. brewer's yeast, have long been known to be capable of converting the fermentable sugars to alcohols such as methanol and ethanol and bacteria such as Clostridium have also long been known to be capable of converting fermentable sugars to alcohols such as ethanol, propanol and butanol. The alcohols produced may be separated off from the fermentation mixture (for example by distillation) and used, e.g. as fuels.

The production of ethanol for use as a fuel in this way has recently received much attention; however butanol, more specifically n-butanol, is perhaps a more attractive option as a fuel since it is more readily mixed with conventional liquid hydrocarbon fuels and since its calorific yield on combustion is higher than that of ethanol.

The cellulose degradation to fermentable sugars is typically effected by hydrolysis using dilute or concentrated mineral acids, for example sulphuric or hydrochloric acids. While acid hydrolysis is very efficient, before the subsequent fermentation step can be carried out the hydrolysate must be neutralized, e.g. by addition of calcium carbonate, and the acid and neutralizing base contribute significantly to the costs of alcohol production from cellulosic starting material.

The fermentable sugars, the product of the polysaccharide (cellulose) degradation, are of course feedstuffs for many if not most microorganisms, including those which do not generate alcohols as metabolic products. Accordingly it is typically necessary in conventional techniques to sterilize the sugars in order to maximize alcohol production by the alcohol producing microorganism, e.g. the brewer's yeast.

We have now found that alcohol production from cellulosic raw materials may be made more efficient by the use of thermophilic microorganisms at a temperature of at least 45° C., especially at least 50° C., particularly at least 60° C., e.g. 60 to 80° C., for both cellulose to fermentable sugar degradation and for fermentable sugar to alcohol conversion. In this way the need for sterilization is reduced or avoided and the need for acid hydrolysis is avoided. Moreover, particularly where ethanol or methanol is being produced, the alcohol may be withdrawn from the fermentation mixture during fermentation so driving the fermentation reaction to a higher alcohol yield.

Thus viewed from one aspect the invention provides a process for the production of an alcohol which comprises cleaving polysaccharides in a cellulosic material in an aqueous medium at a temperature of at least 45° C. using a thermophilic microorganism to yield fermentable sugars, fermenting an aqueous solution of said sugars at a temperature of at least 45° C. with a thermophilic microorganism to yield an alcohol or alkanoate, if necessary reducing said alkanoate to yield an alcohol, and removing said alcohol from said aqueous solution.

Conversion of fermentable sugars by the microorganism may yield an alcohol. Alternatively however, especially where butanol is to be prepared, microorganisms may be used which yield an alkanoate (e.g. butyrate or acetate) instead of or in addition to an alcohol. The alkanoate can be reduced, e.g. hydrogenated, to form the corresponding alcohol with or without first being separated out of the fermentation mixture. In general however, the processes of the invention will preferably involve fermentation to yield an alcohol and will not involve alkanoate reduction.

By “thermophilic” is meant herein that the microorganism must be capable of proliferating in aqueous solution at a temperature of at least 45° C. over a prolonged period, e.g. at least 10 hours, preferably a temperature of at least 50° C., more preferably at least 60° C., especially 60 to 80° C.

The fermentation and cellulose breakdown steps in the process of the invention are preferably performed at a temperature of least 50° C., more preferably at least 60° C., especially 60 to 80° C.

In a particularly preferred embodiment of the process of the invention, polysaccharide to fermentable sugar breakdown and fermentable sugar to alcohol (or to alkanoate) conversion are effected in a single stage using a combination of thermophilic microorganisms. In this embodiment, the different microorganisms may be added to the cellulosic material simultaneously or sequentially and in single steps or repeatedly.

Alcohol (or alkanoate) removal from the fermentation medium may be effected conventionally, e.g. by distillation from the fermentation medium when fermentation has taken place, or more preferably by withdrawing the gas from above the fermenting mixture and condensing the alcohol (or alkanoate) from the withdrawn gas. This is especially preferred where the alcohol to be produced is methanol or, especially. ethanol. In this embodiment, it is especially preferred to cycle the gas from above the fermentation mixture, through a cooled condenser and back into or to above the fermentation mixture. Apparatus for performing such an alcohol removal is itself novel and forms a further aspect of the invention. Thus viewed from this aspect the invention provides apparatus for alcohol collection comprising: a fermentation vessel having a heater; a condenser; and a gas conduit from said vessel to said condenser and back to said vessel. The apparatus is preferably provided with a pump to facilitate gas flow from the fermentation vessel to the condenser, with a cooler (e.g. a cooling jacket) for the condenser, and with an outlet port in the condenser for removal of condensed liquid therefrom.

Viewed from another aspect the invention provides a process for the production of an alcohol (e.g. ethanol, methanol or butanol) which process comprises fermenting an aqueous solution of fermentable sugars (e.g. hexoses and/or pentoses) with a microorganism capable of converting said sugars to alcohol in a fermentation vessel at a temperature of 60 to 80° C. under a gaseous atmosphere, and during the fermentation withdrawing gas from said atmosphere into a cooled condenser to cause entrained alcohol to condense out.

In this process, an inert gas, e.g. nitrogen, hydrogen or carbon dioxide, is preferably passed through the fermentation mixture so as to increase the alcohol content of the gaseous atmosphere removed from the fermentation vessel. This gas may typically be the gas withdrawn from the fermentation vessel subsequent to its passage through the condenser. Gas withdrawal moreover moves the alcohol production reaction equilibrium so as to increase alcohol production. Similarly, alcohol may be removed from the fermentation medium so as to drive the reaction to higher alcohol production by the use of selective membranes or pervapouration techniques.

The cellulose breakdown to fermentable sugars and the conversion of the fermentable sugars to alcohols according to the invention may be done in a single bioreactor or in a two-step reactor system and the microorganism or microorganism cocktail used to achieve the conversions from cellulose to sugars and sugars to alcohol may be the same or different.

The microorganisms used for polysaccharide breakdown may be any thermophilic microorganism capable of achieving this. Suitable microorganisms may be found in the hot centre of any compost heap. Highly thermophilic microorganisms may be isolated by cultivating a sample from such an environment at successively higher temperatures, e.g. raising the temperature in 5 C.° increments from 35° C. to the desired operating temperatures. Alternatively, and generally more preferably, such organisms may be isolated from source materials by incubating at the desired, elevated operating temperatures. Examples of useful microorganism species include Clostridium thermocellum, C. stercorarum, C. straminisolvens, and C. thermoamylolyticum, especially C. thermocellum DSM1237, C. stercorarum DSM8532, C. straminisolvens DSM16021, and C. thermoamylolyticum DSM2335 (see also Ozkan et al., J. Ind. Micribiol. & Biotech. 27:275-280 (2001)).

Suitable cellulose breakdown promoting microorganisms include those producing cellulases, hydrolases, laccases and/or peroxidases. Clostridium strains having cellulose degrading enzymes are known (see for example Sakka et al, Agricultural and Biological Chemistry 53:905-910 (1989), and Kato et al Int. J. Syst. Evol. Microbiol. 54:2043-2047 (2004)), and may conveniently be used in the present invention.

If desired, the microorganisms used for cellulose breakdown may include organisms capable of lignin degradation. Otherwise lignin may be removed from the process and used as a fuel to provide part of the energy required for the overall process.

The microorganisms used for fermentable sugar to alcohol conversion may also be any thermophilic microorganisms capable of achieving this. Thermophilic microorganisms for alcohol production may be identified by cultivating candidates, e.g. yeasts or Clostridium strains at the desired operating temperatures of the process of the invention, or alternately but less preferably at successively higher temperatures from a lower but elevated temperature up to the desired operating temperatures, e.g. raising the temperature in 5 C.° increments from 40° C. to the desired operating temperatures. Thermophilic Clostridium strains are already known, e.g. C. thermocellum, C. fervidus, C. thermosulfurogenes, C. thermohydrosulfuricum, C. caminithermale, C. stercorarium, C. thermolacticum, C. thermocopriae, C. straminisolvens, C. thermopapyroliticum, C. thermobutyricum, C. thermopalmarium and C. thermosaccharolyticum (see for example Mendez et al., Int. J. Syst. Bacteriol. 41:281-283 (1991), Jin et al., Int. J. Syst. Bacteriol. 38:279-281 (1998), Le Ruyet et al., Syst. Appl. Microbiol. 6:196-202 (1985), Madden, Int. J. Syst. Bacteriol. 33:837-840 (1983), Hyun et al., J. Bacteriol. 156:1332-1337 (1983), Ng et al., Arch. Microbiol. 114:1-7 (1977), Wigel et al., J. Ind. Microbiol. and Biotech. 24:7-13 (2000), Lawson et al., Syst. Appl. Microbiol. 14:135-139 (1991), Hollaus et al., Arch. Micriobiol. 86:129-146 (1972) and McClung, J. Bacteriol. 29:189-202 (1935)). One such strain is deposited at the South American Biotechnology and Applied Microbiology Culture Collection as UBA 305. Other microorganism species capable of fermenting at least some of the sugars to form useful alkanols, especially ethanol and butanol, include Thermohydrogenium kirishiense (see Zacharova et al., Arch. Microbiol. 160:492-497 (1993)), Thermobacteriodes acetoethylicus (see Ben-Basset et al., Arch. Microbiol. 128:365-370 (1981)), Thermoanaerobium lactoethylicum (see Kondratieva et al., Arch. Microbiol. 151:117-122 (1989)), Butyribacterium methylotrophicum (see Wordet et al., Fuel 70 (1990)), and, less preferably, Pyrodictium abyssi (see Pley et al., Syst. Appl. Microbiol. 14:245-253 (1991)) and Hyperthermus butylicus (see Zillig et al., J. Bacteriol. 172:3959-3965 (1990)).

In a preferred embodiment of the invention, the microorganism used for alcohol production is a genetically modified form of a microorganism capable of producing an alkanoate from an alkanol bioprecursor, the genetic modification being to knock out (i.e. disable) or delete a gene responsible for the alkanol bioprecursor to alkanoate conversion. In the case of Clostridium for example this may involve knocking out or deleting the gene(s) responsible for converting acetyl-CoA to acetate and/or for converting butyryl-CoA to butyrate or by potentiating or reinforcing the genes responsible for converting acetyl-CoA to ethanol or butyryl-CoA to butanol. This may readily be achieved by conventional techniques, such as gene disruption, knock-out mutagenesis or negative enzyme evolution. Likewise, the microorganism may be transfected with a plasmid capable of generating anti-sense mRNA to block production of undesired enzymes, e.g. enzymes promoting ethanol production when butanol production is desired, and the like. it is also particularly preferred to utilize a genetically modified form of a microorganism capable of producing both ethanol and butanol, the genetic modification being to knock out (i.e. disable) or delete a gene responsible for the ethanol or the butanol production. In the case of Clostridium for example this may involve knocking out or deleting the gene(s) responsible for converting acetyl-CoA to ethanol or for converting butyryl-CoA to butanol or for converting acetyl-CoA to butyryl-CoA or by potentiating or reinforcing the genes responsible for converting acetyl-CoA to ethanol or butyryl-CoA to butanol. This again may easily be achieved by conventional techniques.

Thus, typically, supplementation of genes for the enzymes acetaldehyde dehydrogenase or ethanol dehydrogenase may lead to enhanced ethanol production as may deletion, disablement or suppression of the genes for the enzymes phosphotransacetylase, acetate kinase, thiolase, acetoacetyl-CoA:acetate/butyrate coenzyme-A transferase, acetoacetate decarboxylase, 3-hydroxybutyryl-CoA dehydrogenase, crotonase, butyryl-CoA dehydrogenase, phosphotransbutyrylase, butyrate kinase, butyraldehyde dehydrogenase, aldehyde/alcohol dehydrogenase E, and butanol dehydrogenase or disablement of these enzymes or disablement of RNA coding therefor using antisense RNA. Likewise, supplementation of genes for the enzymes butyraldehyde dehydrogenase, aldehyde/alcohol dehydrogenase E, or butanol dehydrogenase, and optionally thiolase, 3-hydroxybutyryl-CoA dehydrogenase, crotonase, and butyryl-CoA dehydrogenase may lead to enhanced butanol production as may deletion, disablement or suppression of the genes for the enzymes acetaldehyde dehydrogenase, ethanol dehydrogenase, phosphotransacetylase, acetate kinase, acetoacetyl-CoA:acetate/butyrate coenzyme-A transferase, acetoacetate decarboxylase, phosphotransbutyrylase, and butyrate kinase or disablement of these enzymes or disablement of RNA coding therefor using antisense RNA.

Suitable starting species for such manipulation to enhance butanol production include Clostridium thermobutyricum, C. thermopalmarium, C. thermocopriae, C. thermosaccharolyticum, Eubacterium limosum, Thermohydrogenium kirishiense, Pseudoamibacter alactolyticus, Thermobacteriodes acetoethylicus, Thermoanaerobium lactyloethylicum, Thermoproteus uzoniensis, Pyrodictium abyssi, Hyperthermus butylicus, Thermococcus stetteri and Butyribacterium methylotrophicum.

Thus, for example, butanol production may be enhanced in Clostridium sp. by transformation with plasmids pCAAD or pTHAAD which carry the gene aad (see Nair et al., J. Bacteriol. 176:871-885 (1994) and J. Bacteriol. 176:5843-5846 (1994) and Green et al., Biotech. and Bioeng. 58:215-221 (1998)). Other plasmids suitable for use in introducing genes into a broad range of Clostridium species are discussed for example by Blaschek et al. in FEMS Microbiology Reviews 17:349-356 (1995). Antisense RNA may likewise be used to suppress the effects of genes which direct production away from the desired alkanols (see Tummala et al., in J. Bacteriol. 185:1923-1934 (2003)). Classical mutagenesis may of course also be used—authors such as Annous et al., in Appl. Env. Microbiol. 57:2544-2548 (1991) have reported successful use of classical mutagenesis in boosting butanol production by Clostridium sp.

Such genetically modified forms of thermophilic alcohol/alkanoate producing microorganisms are novel and form a further aspect of the invention. Viewed from this aspect the invention provides a thermophilic microorganism, e.g. capable of proliferating at temperatures in excess of 45° C., especially in excess of 50° C., particularly in excess of 60° C., preferably of the species Clostridium, capable of metabolizing hexoses and/or pentoses to produce ethanol and/or butanol wherein a gene coding for an enzyme operative to convert acetyl-CoA to acetate, to butyryl-CoA or to ethanol or a gene coding for an enzyme operative to convert butyryl-CoA to butyrate is disabled or deleted. Particularly preferably, at least two such genes, in particular two or three such genes, are disabled or deleted. Disablement or deletion in this context includes transformation to generate antisense RNA which reduces or prevents successful gene expression.

Examples of microorganisms useful in the process of the invention for generating alcohols or alkanoates, for breaking down biomass to produce a substrate for alcohol or alkanoate generation or as starting materials for modification as described above include: Clostridium acetobutylicum (grows at 37° C.); C. beijerinckii (grows at 35° C.); C. josui (breaks down cellubiose, esculin and xylose, grows at 45° C., pH 7.0); C. thermocopriae (breaks down cellulose and a variety of sugars, grows at 60° C., pH 6.5-7.3); C. thermosaccharolyticum (breaks down sucrose, dextrin, and pectin, grows at 55-62° C.); C. thermohydrosulfuricum (breaks down starch, cellubiose, glucose, xylose and soluble sugars, grows at 68° C., pH 6.9-7.5); C. thermobutyricum (breaks down soluble sugars, grows at 55° C., pH 6.8-7.1); C. thermopalmarium (breaks down sugars, grows at 55° C., pH 6.6); C. carboxidivorans (breaks down glucose, starch, cellulose, cellubiose and pectin, grows at 38° C., pH 6.2); Thermobacteroides acetoethylicus (breaks down starch, glucose and other soluble sugars, grows at 65° C., pH 5.5-8.5); Thermoanaerobium lactoethylicum (breaks down starch, glucose and other sugars, grows at 65° C., pH 7.0); Pyrodictium abyssi (breaks down starch and gelatin, grows at 97° C., pH 5.5); Thermococcus stetteri (breaks down peptone, starch and peptin, grows at 73-77° C., pH 6.5); Oxobacter pfennigii (grows at 36-38° C., pH 7.3); Butyribacterium methylotrophicum (grows at 37° C., pH 6.0); and Burkholderia xenovorans).

Further examples of microorganisms useful in the process of the invention for generating butanol or butanoates, for breaking down biomass to produce a substrate for butanol or butanoate generation or as starting materials for modification as described above include: Clostridium thermosaccharolyticum ATCC 7956 (butanol-producing, grows at 45° C.); C. thermopalmarium DSM 5974 (butyrate-producing, grows at 55° C.); C. carboxidivorans ATCC BAA-624 (butanol-producing, grows at up to 40° C.); Thermoanaerobacter acetoethylicus ATCC 33265 (butyrate-producing, grows at 60° C.); Thermococcus stetteri DSM 5262 (isobutyrate-producing, grows at 75° C.); and Oxobacter pfennigii DSM 3222 (butyrate-producing, grows at 37° C.). All of these have an acid generating phase followed by a solvent (butanol) generating phase; however the initial butyrate generation is especially effective for C. carboxidivorans, T. acetoethylicus and, especially, O. pfennigii.

In an especially preferred embodiment of the invention, the microorganisms used for polysaccharide to sugar breakdown and for sugar to alcohol conversion are of the same species, e.g. Clostridium.

Where the microorganisms used are anaerobic, the relevant process step is preferably performed under an oxygen-free or oxygen-depleted atmosphere (e.g. containing 0 to 10 mole % oxygen, preferably 0 to 5 mole %, especially 0 to 2 mole %). In this way competition for nutritional resources by aerobic microorganisms is restricted. Particularly preferably the composition being treated, e.g. an aqueous cellulosic material or an aqueous sugar solution, is treated to reduce oxygen content, for example by exposure to reduced pressure or by flushing with a non-oxygen gas such as nitrogen, carbon dioxide or a noble gas.

As mentioned above, when methanol or ethanol is being produced this may be removed from the gas above the fermentation mixture during fermentation. Alternatively, irrespective of the nature of the alcohol being produced, the alcohol product may be removed from the fermented mixture by distillation. In another preferred embodiment, alcohol may be removed during or after fermentation, preferably during fermentation, by contacting the aqueous fermentation mixture with a water-immiscible organic liquid such as for example a liquid hydrocarbon. The alcohol may be removed from the organic liquid by distillation or the liquid with entrained alcohol may be used directly as a fuel. Once again, if alcohol is extracted in this way during fermentation, the fermentation reaction may be driven to increase overall alcohol yield.

The raw starting material for the process of the invention may be any convenient cellulosic material. Preferably the material comprises wood (e.g. wood pulp), paper, forest debris, grass, straw, corn husks or the like. While seeds or nuts as such are not a particularly desirable starting material, seed or nut waste from pressing for plant oil may conveniently be used.

Advantageously, the raw material is subjected to chemical and/or physical pretreatment to accelerate subsequent cellulose breakdown, e.g. maceration or steam treatment.

The invention will now be illustrated further with reference to the following non-limiting Examples and the attached drawing in which:

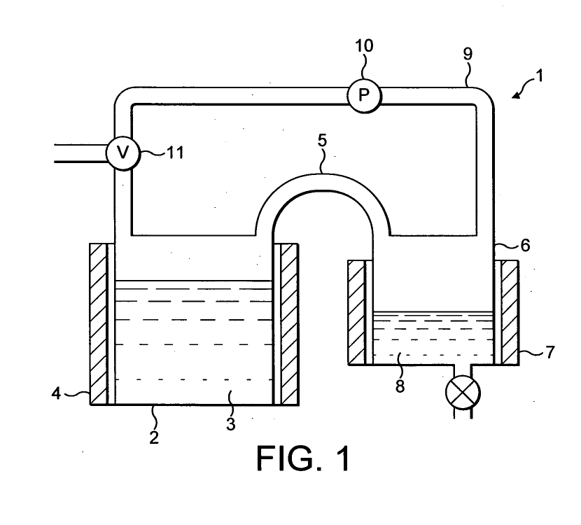

FIG. 1 is a schematic diagram of apparatus according to the invention.

Referring to FIG. 1, there is shown an apparatus 1 for the production of an alcohol such as ethanol. Fermentation vessel 2, containing fermentable sugars in aqueous solution 3 produced by cellulose degradation, is provided with a heating jacket 4 to maintain the solution temperature at 70±5° C. Conduit 5 leads from vessel 2 to condenser unit 6 which is provided with a water cooling jacket 7 to maintain a temperature close to ambient and so cause condensation of alcohol 8. Return conduit 9 leads back from condenser unit 6 to fermentation vessel 2 via a pump 10. Valve 11 is provided in return conduit 9 to introduce air or nitrogen or to reduce overpressure in the apparatus as desired or required.

### EXAMPLE 1

### Polysaccharide Breakdown

Cellulosic material, in this case wood pulp, is preheated by stream explosion to facilitate subsequent microbial degradation. To the pretreated pulp is added an aqueous inoculate from a compost heap. The mixture is maintained at 60° C. for three days.

### EXAMPLE 2

### Alcohol Production

The product of Example 1 is inoculated with a butanol producing strain of Clostridium and incubated at 60° C. for two days under a nitrogen atmosphere whereafter the butanol produced is recovered by distillation.

1. A process for the production of an alcohol which comprises cleaving polysaccharides in a cellulosic material in an aqueous medium at a temperature of at least 45° C. using a thermophilic microorganism to yield fermentable sugars, fermenting an aqueous solution of said sugars at a temperature of at least 45° C. with a thermophilic microorganism to yield an alcohol, and removing said alcohol from said aqueous solution.

2. A process as claimed in claim 1 wherein cleavage and fermentation is effected at a temperature of at least 60° C.

3. A process for the production of an alcohol which process comprises fermenting an aqueous solution of fermentable sugars with a microorganism capable of converting said sugars to alcohol in a fermentation vessel at a temperature of 60 to 80° C. under a gaseous atmosphere, and during the fermentation withdrawing gas from said atmosphere into a cooled condenser to cause entrained alcohol to condense out.

4. A process for the production of an alcohol which process comprises fermenting an aqueous solution of fermentable sugars with a microorganism capable of converting said sugars to alcohol in a fermentation vessel at a temperature of 60 to 80° C. and during the fermentation withdrawing alcohol from said solution.

5. A process as claimed in claim 1 comprising fermenting a said aqueous solution of fermentable sugars with a microorganism capable of converting said sugars to butanol.

6. A thermophilic microorganism capable of metabolizing hexoses and/or pentoses to produce ethanol and/or butanol wherein a gene coding for an enzyme operative to convert acetyl-CoA to acetate, to butyryl-CoA or to ethanol or a gene coding for an enzyme operative to convert butyryl-CoA to butyrate is disabled or deleted.

7. Apparatus for alcohol collection comprising: a fermentation vessel having a heater; a condenser; and a gas conduit from said vessel to said condenser and back to said vessel.

8. A process as claimed in claim 2 comprising fermenting a said aqueous solution of fermentable sugars with a microorganism capable of converting said sugars to butanol.

9. A process as claimed in claim 3 comprising fermenting a said aqueous solution of fermentable sugars with a microorganism capable of converting said sugars to butanol.

10. A process as claimed in claim 4 comprising fermenting a said aqueous solution of fermentable sugars with a microorganism capable of converting said sugars to butanol.
